(19) United States
(12) Patent Application Publication
(10) Pub. No.: US 2006/0167283 A1
(43) Pub. Date: Jul. 27, 2006

(54) Method of preparing cannabidiol from plant material

(76) Inventors: Ian Flockhart, Hull (GB); Gary William Wheatley, Salisbury (GB); Su Dring, Hull (GB); Lesley Archer, Yorkshire (GB)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 10/528,957

(22) PCT Filed: Sep. 23, 2003
(86) PCT No.: PCT/GB03/04086

§ 371 Date: Mar. 22, 2005

(30) Foreign Application Priority Data
Sep. 23, 2002 (GB) 0222078.8

(51) Int. Cl. C07D 311/80

(52) U.S. Cl. 549/390

(57) ABSTRACT

The invention relates to methods of preparing cannabidiol in substantially pure form starting from plant material. Also described are substantially pure preparations of cannabidiol having a chromatographic purity of 95% or greater.

### FIELD OF THE INVENTION

The invention relates to methods of preparing cannabidiol in substantially pure form starting from plant material.

### BACKGROUND TO THE INVENTION

Cannabis has been used medicinally for many years, and in Victorian times was a widely used component of prescription medicines. It was used as a hypnotic sedative for the treatment of “hysteria, delirium, epilepsy, nervous insomnia, migraine, pain and dysmenorrhoea”. Historically, cannabis was regarded by many physicians as unique; having the ability to counteract pain resistant to opioid analgesics, in conditions such as spinal cord injury, and other forms of neuropathic pain including pain and spasm in multiple sclerosis.

The use of cannabis continued until the middle of the twentieth century, when the recreational use of cannabis prompted legislation which resulted in the prohibition of its use. The utility of cannabis as a prescription medicine is now being re-evaluated. The discovery of specific cannabinoid receptors and new methods of administration have made it possible to extend the use of cannabis-based medicines to historic and novel indications.

The principle cannabinoid components present in herbal cannabis are the cannabinoid acids Δ⁹ tetrahydrocannabinolic acid (Δ⁹ THCA) and cannabidiolic acid (CBDA), with small amounts of the corresponding neutral cannabinoids, respectively Δ⁹ tetrahydrocannabinol (Δ⁹ THC) and cannabidiol (CBD).

Cannabidiol (CBD) was formerly regarded as an inactive constituent, however there is emerging evidence that it has pharmacological activity, which is different from that of Δ⁹ THC in several respects.

Wider studies of the pharmacology of CBD are needed in order to fully explore its pharmaceutical potential. Thus, there is a need for substantially pure preparations of CBD for use in such studies.

WO 02/064109 describes a general method for obtaining whole extracts from cannabis plant material.

WO 02/32420 discloses a process for preparing, for example, Δ⁹-THC from plant material. It utilises CO₂ extraction and ethanol precipitation to obtain “primary extracts” containing Δ⁹-THC and CBD, with reduced amounts of, for example, monoterpenes, sesquiterpenes, hydrocarbons, alkaloids, flavonoids and chlorophylls. The CBD is then converted to Δ⁹-THC by a catalysing reaction. The cannabinoids make up only approximately two-thirds of the composition and are therefore not substantially pure.

ODCCP Bulletin on Narcotics (1976, Issue 4) discloses a method of isolating CBD, THC and CBN using preparative gas chromatography.

ODCCP Bulletin on Narcotics (1978, Issue 4) describes a multi-solvent extraction process using petroleum ether and methanol.

Journal of Pharmacy and Pharmacology (1977, 27(5)) discloses the use of various solvents as extraction medium for solubilising cannabinoids.

A review of methods disclosed in these prior art documents has determined that none of the processes are selective for CBD as is described by the subject of the present invention, all may include significant amounts of psychoactive cannabinoids such as THC and CBN.

U.S. Pat. No. 2,304,669 discloses a multiple step method for isolating CBD from plant extracts, the process involves the treatment of oil derived from cannabis plants with 3,5-dinitrobenzoylchloride to form cannabidiol bis-3,5-dinitrobenzoate, separating this mixture from the oil and then subjecting this benzoate ester to ammonolysis to produce purified cannabidiol.

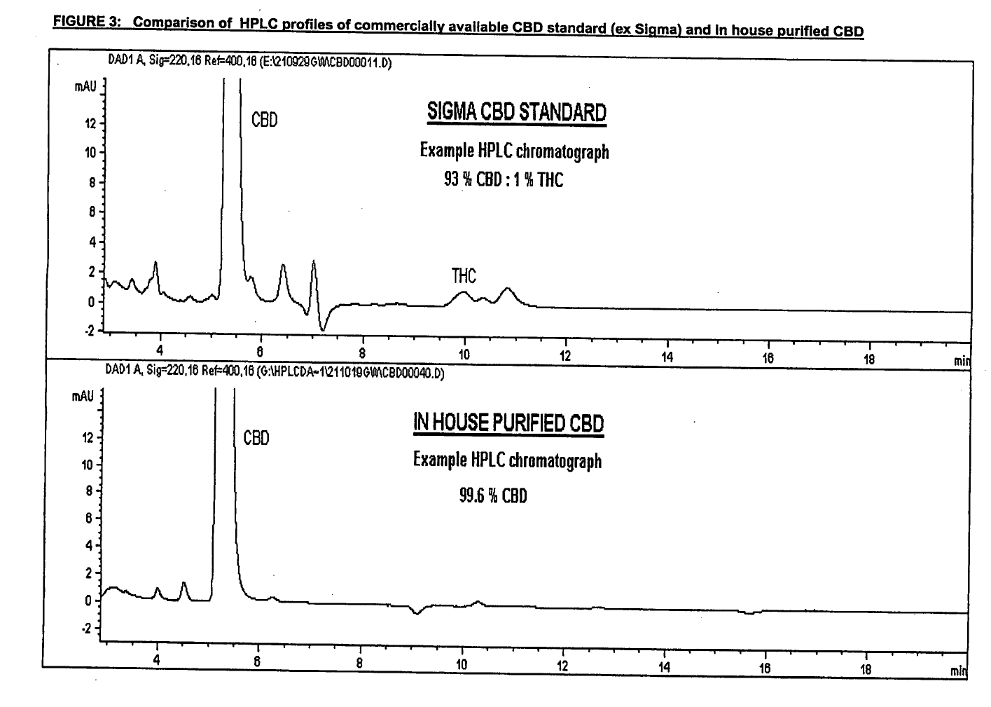

Cannabidiol solution in methanol is currently available from Sigma-Aldrich, but the comparative tests shown here in FIG. 3 show that it is not substantially pure.

Synthetic forms of cannabidiol are commercially available (e.g. from Sigma Corp.) but are prohibitively expensive. Furthermore, HPLC analysis reveals the presence of significant amounts of Δ⁹ THC (typically around 1%) in the commercially available preparations of cannabidiol.

Thus, there is a need for a method of production of cannabidiol which is inexpensive and yet capable of yielding substantially pure cannabidiol, particularly cannabidiol containing less Δ⁹ THC than the currently available preparations. Furthermore, cannabidiol has pharmaceutical potential, thus there is a strong need to produce cannabidiol without psychoactive contaminants such as THC or CBN. Such a method for the production of cannabidiol should preferably be easy, cheap and capable of scale-up.

The inventors have therefore focussed on the purification of CBD from plant material and have developed a process for the preparation of substantially pure crystalline CBD from plant material.

### SUMMARY OF THE INVENTION

In a first aspect the invention provides a selective method of obtaining substantially pure cannabidiol (CBD) from plant material, which method comprises obtaining a cannabidiol-containing extract of the plant material, dissolving the extract in a solvent to form a solution, removing insoluble material from this solution and evaporating the solvent from the solution to obtain substantially pure cannabidiol.

In a second aspect the invention provides a substantially pure preparation of cannabidiol (CBD) having a chromatographic purity of 95% or greater, preferably 96% or greater, more preferably 97% or greater, more preferably 98% or greater, more preferably 99% or greater and most preferably 99.5% or greater by area normalisation of an HPLC profile.

### DESCRIPTION OF THE INVENTION

The invention relates to a purification process for selectively preparing substantially pure cannabidiol (CBD) from plant material.

A “selective” method is defined as one which preferentially discerns CBD from a partially crude mixture of cannabinoids as are often found in an extract from cannabis plant material.

A “substantially pure” preparation of cannabidiol (CBD) is defined as a preparation having a chromatographic purity of 95% or greater, more preferably 96% or greater, more preferably 97% or greater, more preferably 98% or greater, more preferably 99% or greater, and most preferably 99.5% or greater as determined by area normalisation of an HPLC profile.

The process of the invention involves obtaining a cannabidiol-containing extract from a plant material, dissolving the extract in a solvent to form a solution, removing insoluble material from this solution (preferably by filtration) and evaporating the solvent from the solution (for example by rotary evaporation) to obtain substantially pure cannabidiol.

In a preferred embodiment substantially pure cannabidiol is obtained in crystalline form.

The solvent used to re-dissolve the cannabidiol-containing extract is preferably a non-polar liquid solvent. The purpose of this solvent treatment step is to remove non-cannabidiol impurities to leave a substantially pure preparation of cannabidiol. Suitable non-polar solvents therefore include essentially any non-polar solvents which are substantially less polar than cannabidiol, such that impurities which are more polar than cannabidiol are removed by treatment with the solvent. Preferred non-polar solvents include, but are not limited to, C5-C12 straight chain or branched chain alkanes, or carbonate esters of C1-C12 alcohols. It is preferred to use the more volatile C5-C12 alkanes, as they are more easily removed by evaporation. Particularly preferred solvents include pentane (preferably n-pentane), hexane (preferably n-hexane) and propyl carbonate.

The method of the invention may be used to prepare substantially pure cannabidiol from any plant material known to contain cannabidiol (CBD), or the corresponding cannabinoid acid cannabidiolic acid (CBDA). Most typically, but not necessarily, the “plant material” will be derived from one or more cannabis plants.

The term “plant material” encompasses a plant or plant part (e.g. bark, wood, leaves, stems, roots, flowers, fruits, seeds, berries or parts thereof) as well as exudates, and includes material falling within the definition of “botanical raw material” in the Guidance for Industry Botanical Drug Products Draft Guidance, August 2000, US Department of Health and Human Services, Food and Drug Administration Centre for Drug Evaluation and Research.

The term “cannabis plant(s)” encompasses wild type Cannabis sativa and also variants thereof, including cannabis chemovars (varieties characterised by virtue of chemical composition) which naturally contain different amounts of the individual cannabinoids, also Cannabis sativa subspecies indica including the variants var. indica and var.kafiristanica, Cannabis indica and also plants which are the result of genetic crosses, self-crosses or hybrids thereof. The term “cannabis plant material” is to be interpreted accordingly as encompassing plant material derived from one or more cannabis plants. For the avoidance of doubt it is hereby stated that “cannabis plant material” includes herbal cannabis and dried cannabis biomass.

It is preferred to use cannabis plant material derived from cannabis plants having a relatively high content of CBD (as CBDA and/or CBD). With the use of standard selective breeding techniques the present inventors have developed cannabis varieties (chemovars) having a CBDA/CBD content of >90% of the total cannabinoid content.

If the plant material from which CBD is to be prepared contains significant amounts of the cannabinoid acid CBDA then the plant material may be subjected to a decarboxylation step to convert CBDA to the free cannabinoid CBD. This is preferably carried out prior to preparation of the CBD-containing plant extract or may form part of this extraction process.

Decarboxylation is preferably carried out by heating the plant material to a defined temperature for a suitable length of time. Decarboxylation of cannabinoid acids is a function of time and temperature, thus at higher temperatures a shorter period of time will be taken for complete decarboxylation of a given amount of cannabinoid acid.

Preferably, decarboxylation is carried out in a multi-step heating process in which the plant material is:
i) heated to a first temperature for a first (relatively short) time period to evaporate off retained water and allow for uniform heating of the plant material; and
ii) the temperature is increased to a second temperature for a second time period (typically longer than the first time period) until at least 95% conversion of the acid cannabinoids to their neutral form has occurred.

Preferably the first step is conducted at a temperature in the range of from 100° C. to 110° C. for 10-20 min. More preferably the first temperature is about 105° C. and the first time period is about 15 minutes.

Optimum times and temperatures for the second step may vary depending on the nature of the plant material, and more particularly on the cannabinoid which it is intended to isolate from the plant material, and may be easily determined by routine experiment. Suitable conditions may include, for example, a temperature in the range of from 115° C. to 125° C. for a time period in the range of from 45 to 75 minutes (typically 120° C. for 60 minutes), or a temperature in the range of from 135° C. to 145° C., for a time period in the range of from 15 to 45 minutes.

If the plant material is derived from cannabis plants having a high CBD content (defined as >90% CBD as a percentage of total cannabinoid content), the second temperature is preferably in the range of from 115° C. to 125° C., preferably about 120° C. and the second time period is in the range of from 45 to 75 minutes, preferably about 60 minutes. More preferably the second temperature is in the range of from 135° C. to 145° C., preferably 140° C. and the second time period is in the range of from 15 to 45 minutes, preferably about 30 minutes. In another embodiment, most preferred for a mass of plant material greater than 4 kg, the second temperature is in the range of from 140° C. to 150° C., preferably 145° C. and the second time period is in the range of from 55-90 minutes. The exact figures, particularly time, may vary slightly with increased mass. This should be taken into account when scaling up the process to an industrial manufacturing scale.

Where the starting “plant material” is freshly harvested or “wet” plant material it may be subjected to a drying step to remove excess moisture prior to step (i). For convenience, decarboxylation and drying may be combined in a single heating step or in a multi-step heating process, as described above.

The “cannabidiol-containing extract” is preferably a botanical drug substance prepared from plant material, or an ethanolic solution of such a botanical drug substance. In the context of this application a “botanical drug substance” is defined as an extract derived from plant material, which extract fulfils the definition of “botanical drug substance” provided in the Guidance for Industry Botanical Drug Products Draft Guidance, August 2000, US Department of Health and Human Services, Food and Drug Administration Centre for Drug Evaluation and Research of: “A drug substance derived from one or more plants, algae, or macroscopic fungi. It is prepared from botanical raw materials by one or more of the following processes: pulverisation, decoction, expression, aqueous extraction, ethanolic extraction, or other similar processes.”

“Botanical drug substances” derived from cannabis plants include primary extracts prepared by such processes as, for example, maceration, percolation, and solvent extraction. Solvent extraction may be carried out using essentially any solvent that dissolves cannabinoids/cannabinoid acids, such as for example C1 to C5 alcohols (e.g. ethanol, methanol), C5-C12 alkanes (e.g. hexane), Norflurane (HFA134a), HFA227 and carbon dioxide. When solvents such as those listed above are used, the resultant extract typically contains non-specific lipid-soluble material. This can be removed by a variety of processes including “winterisation”, which involves chilling to −20° C. followed by filtration to remove waxy ballast, extraction with liquid carbon dioxide and by distillation. General protocols for the preparation of botanical drug substances from cannabis plant material are described in the applicant's published International patent application WO 02/064109.

The botanical drug substance is preferably obtained by carbon dioxide (CO₂) extraction followed by a secondary extraction, e.g. an ethanolic precipitation, to remove a substantial proportion of non-cannabinoid materials, e.g. waxes, wax esters and glycerides, unsaturated fatty acid residues, terpenes, carotenes, and flavenoids and other ballast. Most preferably the botanical drug substance is produced by a process comprising extraction with liquid CO₂, under sub-critical or super-critical conditions, and then a further extraction, preferably an ethanolic precipitation, to remove significant amounts of ballast.

The resulting ethanolic BDS solution may be subjected to further treatment with activated charcoal. Conveniently, this may be achieved by passing the ethanolic BDS solution down a column of activated charcoal.

Thus, in the most preferred embodiment the botanical drug substance is prepared according to a process comprising the following steps:
i) decarboxylation of the plant material,
ii) extraction with liquid CO₂ (most preferably under sub-critical conditions), to produce a crude botanical drug substance,
iii) precipitation with C1-C5 alcohol (preferably ethanol) to reduce the proportion of non-target materials,
iv) removal of the precipitate (preferably by filtration),
v) optional treatment with activated charcoal, and
vi) evaporation to remove C1-C5 alcohol and water, thereby producing a final botanical drug substance.

A detailed example of such a process is described in the accompanying Examples.

The most preferred embodiment of the purification method of the invention therefore comprises:
i) decarboxylation of the plant material,
ii) extraction with liquid CO₂ (most preferably under sub-critical conditions), to produce a crude botanical drug substance,
iii) precipitation with C1-C5 alcohol (preferably ethanol) to reduce the proportion of non-target materials,
iv) filtration to remove the precipitate,
v) treatment of the resulting solution with activated charcoal,
vi) removal of the C1-C5 alcohol and any water from the solution to produce a CBD-enriched extract,
v) re-dissolving the CBD-enriched extract in pentane,
vi) removal of any insoluble material, if required,
vi) removal of solvent from resulting solution, preferably by evaporation, thereby crystallising cannabidiol.

The process of the invention yields substantially pure cannabidiol of high chromatographic purity, typically as a white crystalline solid.

The invention further relates to a substantially pure preparation of cannabidiol having a chromatographic purity of 95% or greater, more preferably 96% or greater, more preferably 97% or greater, more preferably 98% or greater, preferably 99% or greater, and most preferably 99.5% or greater by area normalisation of an HPLC profile. The preparation is typically a white crystalline solid at room temperature, having a melting point in the range of from 64 to 66° C.

The preparation preferably comprises less than 1%, more preferably less than 0.8%, more preferably less than 0.6%, more preferably less than 0.4%, more preferably less than 0.2% and most preferably less than 0.1% Δ⁹ THC.

The preparation preferably comprises less than 1%, more preferably less than 0.8%, more preferably less than 0.6%, more preferably less than 0.4%, more preferably less than 0.2% and most preferably less than 0.1% CBN.

Most preferably the preparation contains no detectable CBN or Δ⁹ THC, defined as less than 0.1% by HPLC analysis.

The inventors are the first to isolate CBD from plant material at this level of purity in crystalline form. The ability to prepare CBD at a high level of purity will permit further studies of the pharmacology, and hence pharmaceutical utility, of this cannabinoid.

The substantially pure cannabidiol provided by the invention is significantly more pure than the cannabidiol (CBD standard) commercially available from Sigma Corporation (see comparative HPLC analysis, FIG. 3). Of particular significance is the fact that cannabidiol prepared according to the invention contains no detectable Δ⁹ THC (less than 0.1% by HPLC), whereas the Sigma CBD standard contains ˜1% Δ⁹ THC.

The invention will be further understood with reference to the following experimental examples, together with the accompanying Figures, in which:

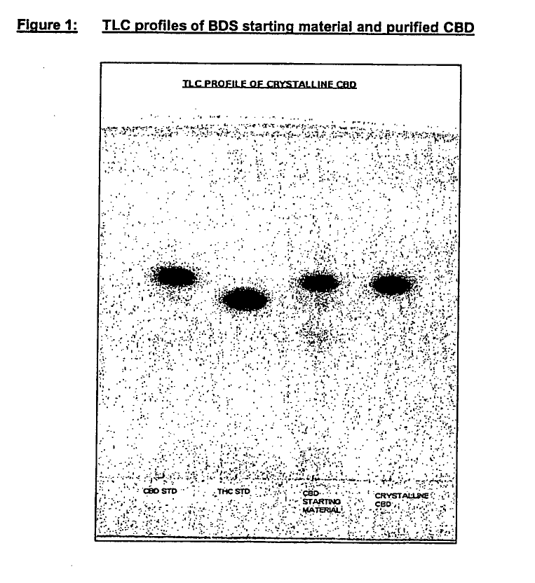

FIG. 1 shows thin layer chromatography (TLC) profiles of purified cannabidiol (CBD), as compared to the starting material (CBD-containing botanical drug substance) and CBD and THC standards (Sigma). Standards were 1 mg/ml CBD (BN 10601/c) or Δ⁹ THC (BN 10601/B) in MeOH, 5 μl of each applied to TLC plate. Samples were 1 mg/ml CBD starting material in MeOH, 5 μl applied to TLC plate, 1 mg/ml crystalline CBD in MeOH, 5 μl applied to TLC plate. Chromatographic conditions: stationary phase SIL G/UV₂₅₄, mobile phase hexane:diethyl ether 80:20, double development, visualisation 0.1% w/v Fast Blue B salt in water.

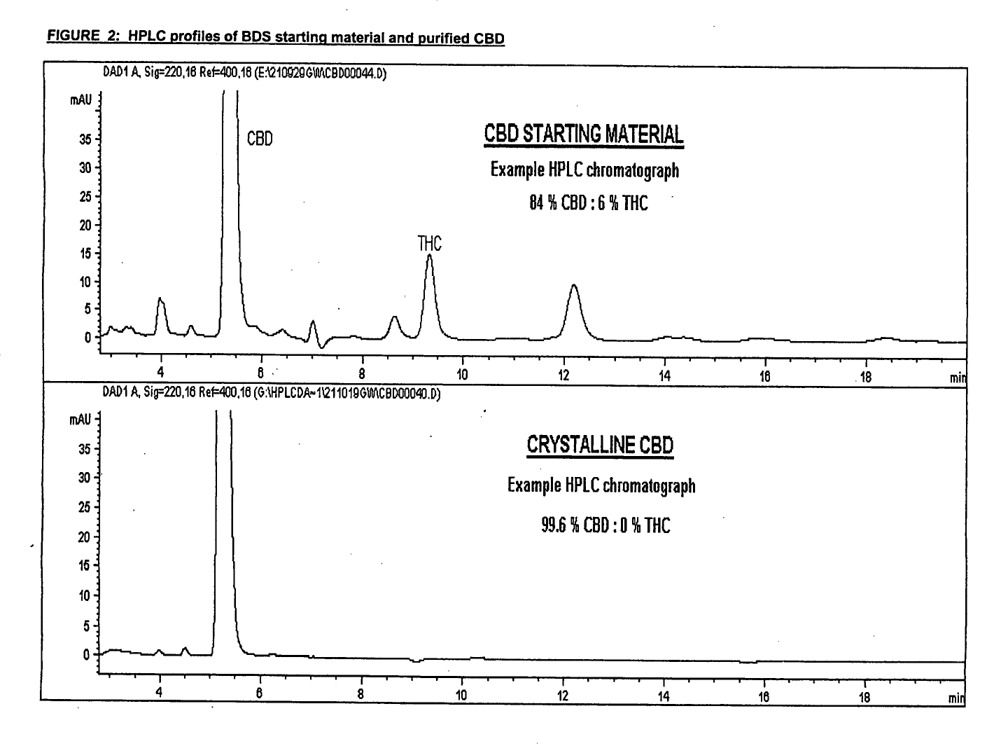

FIG. 2 shows sample HPLC profiles of CBD starting material (botanical drug substance; 86% CBD, 6% THC) and purified, crystalline CBD (99.6% CBD, 0% THC). HPLC was performed as described in the examples.

FIG. 3 shows sample HPLC profiles of purified crystalline CBD (99.6% purity by area normalisation) and Sigma CBD standard (93% CBD, 1% THC).

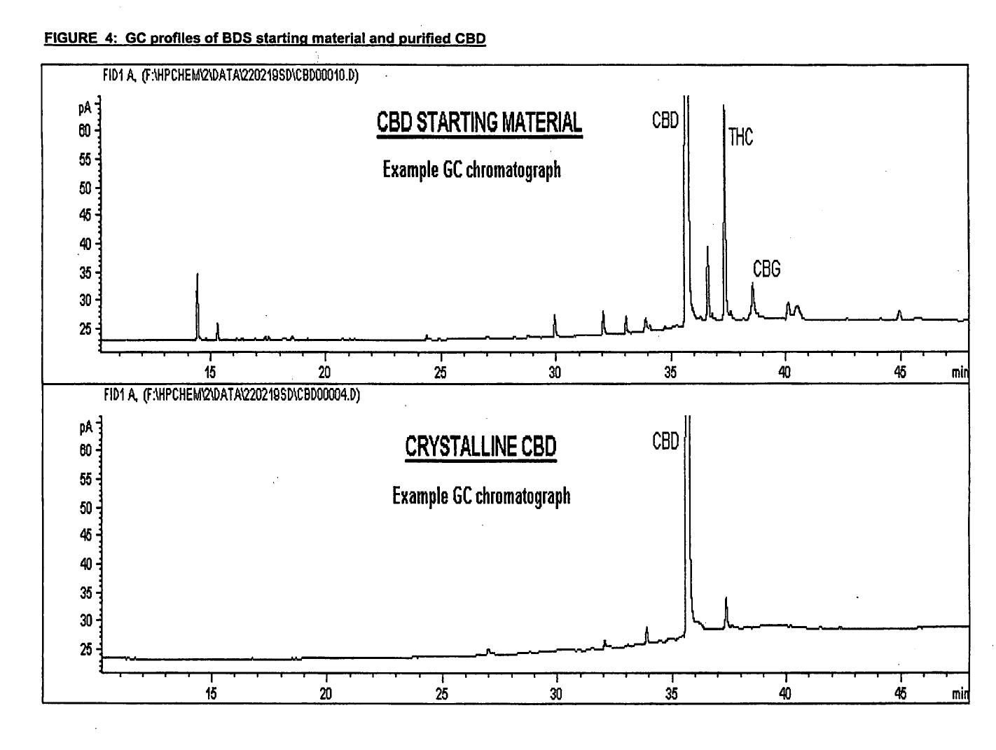

FIG. 4 shows gas chromatographic (GC) analysis of CBD starting material (botanical drug substance) and purified, crystalline CBD.

### EXAMPLE 1

### Purification of CBD

Overview of Process

Starting from freshly harvested plant material the process comprises drying and decarboxylation of the plant material, optional treatment (e.g. milling) of the dried plant material to reduce the particle size (preferably to less than 2000 μm), extraction with liquid carbon dioxide, ethanolic precipitation to reduce the amount of non-target material, clean-up of the crude ethanolic extract by passage through activated charcoal, removal of solvent (ethanol) to produce a CBD-enriched fraction, and re-crystallisation of CBD from pentane.

Plant Material

GW Pharma Ltd has developed distinct varieties of Cannabis plant hybrids to maximise the output of the specific chemical constituents, cannabinoids. A “high CBD” chemovar designated G5 produces >90% total cannabinoid content as CBD (naturally occurring in the plant in the form of CBDA). Alternative “high CBD” varieties can be obtained—see for example, Common cannabinoids phenotypes in 350 stocks of cannabis, Small and Beckstead, Lloydia vol 36b , 1973 p 144-156—and bred using techniques well known to the skilled man to maximise cannabinoid content.

General protocols for growing of medicinal cannabis and for testing the cannabinoid content of cannabis plants are described in the applicant's published International patent application WO 02/064109.

Solvents

All solvents used in the isolation and analysis of CBD (e.g n-pentane) were, unless otherwise stated, of chromatographic or A.R. grade.

Standards

Reference materials from Sigma were used as standards in the analysis of extracts, intermediates and finished products, these were: Δ⁹ THC in methanol BN 10601/B (ca. 1 mg/ml) and CBD in methanol BN 10601/C (ca. 1 mg/ml).

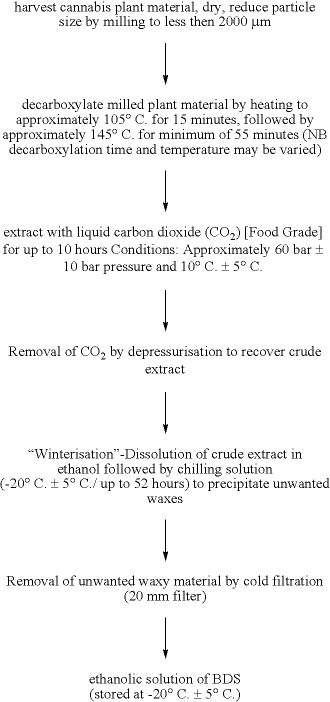

Extraction using liquid CO₂ is carried out under sub-critical conditions at a temperature of approximately 10° C.±5° C. using a pressure of approximately 60 bar+10 bar. Decarboxylated plant material is packed into a single column and exposed to liquid CO₂ under pressure for approximately 8 hours, CO₂ mass flow 1250 kg/hr±20%.

Following depressurisation and venting off of the CO₂ the crude BDS extract is collected into sealed vessels. The crude BDS extract is held at −20° C.±5° C.

The crude BDS extract contains waxes and long chain molecules. Removal is by “winterisation”, whereby the crude BDS extract is warmed to e.g. 40° C.±4° C. to liquefy the material. Ethanol is added in the ratio of 2:1 ethanol volume to weight of crude BDS extract. The ethanolic solution is then cooled to −20° C.±50° C. and held at this temperature for approximately 48 hours.

On completion of the winterisation the precipitate is removed by cold filtration through a 20 μm filter, to give an ethanolic solution of the BDS.

Preliminary charcoal clean-up may be carried out by passing the ethanolic BDS solution (400-500 mg/ml) through a disposable plastic column (130 mm×27 mm i.d) packed with activated charcoal (decolourcarb DCL GDC grade, from Sutcliffe Speakman Carbons, 15.4 g per unit). Absolute ethanol B.P. (Hayman) is used as the solvent.

Ethanol and any water that may be present are removed by rotary evaporation or thin film evaporation under reduced pressure (60° C.±2° C., with vapour at 40° C.±2° C./172 mbar and 72 mbar±4 mbar) to produce a CBD-rich extract.

Solvent Re-Crystallisation

The CBD-rich extract is re-dissolving in a suitable solvent (e.g. n-pentane) and filtered to remove insoluble material. Solvent is them removed, e.g. by rotary evaporation, to produce crystalline CBD. All steps are carried out according to standard laboratory procedures, such as would be known to those skilled in the art.

Product Characteristics

Yield:

3 g of CBD BDS yields approx 1 g of purified CBD.

Characteristics:

White crystalline solid.

Chromatographic purity >99% CBD by area normalization.

Chromatographic purity superior to commercially available CBD Sigma standard (refer to FIGS. 1 and 3).

THC non detected i.e. <0.1%

CBN non detected i.e. <0.1%

Identity confirmed by HPLC, GC and TLC retention behaviour compared to CBD Sigma standard.

Assay vs both Sigma CBD std in range 98.0-102.0%

Melting Point=64-66° C. (literature value=66-67° C.).

HPLC Analysis

The composition of the isolated products may be determined by HPLC analysis.

A typical HPLC assay for Δ⁹ THC, Δ⁹ THCA, CBD, CBDA and CBN may be carried out as follows:

a) Materials and Methods

Chromatography Equipment and Conditions:

Equipment Agilent (HP) 1100 HPLC system with variable wavelength UV detector or diode array detector.

HPLC Column Discovery C8 5 μm 15 cm×0.46 cm

Pre-Column Kingsorb C18 5 μm 3 cm×0.46 cm

Mobile Phase Acetonitrile:Methanol: 0.25% w/v acetic acid (16:7:6 by volume)

Column Temp 25° C.

Flow Rate 1.0 ml min⁻¹

Detection 220 nm 600 mA f.s.d. Second wavelength 310 nm

Injection Volume 10 μl

Run Time 20-25 minutes (may be extended for samples containing small amount of late-eluting peaks)

Elution Order CBD, CBDA, Δ⁹ THCV, CBN, Δ⁹ THC, CBC, Δ⁹ THCA

b) Sample Preparation

Samples of “pure” cannabidiol are diluted in methanol prior to HPLC analysis. Optimal dilutions may be determined empirically.

Herbal cannabis samples are prepared by taking a 100 mg sample and treating this with 5 or 10 ml of Methanol/Chloroform (9/1 w/v). The dispersion is sonicated in a sealed tube for 10 minutes, allowed to cool and an aliquot is centrifuged and suitably diluted with methanol prior to chromatography.

c) Standards

Stock standard solutions of CBD, CBN and Δ⁹ THC in methanol at approximately 1 mg/ml are stored at −20° C. Diluted working standards (0.1 mg/ml for Δ⁹ THC and CBD and 0.01 mg/ml for CBN) are prepared in methanol from the stock standards and stored at −20° C. (maximum period of twelve months after initial preparation). After preparation, standard solutions must be aliquoted into vials to reduce the amount of standard exposed to room temperature. Prior to use in an HPLC sample assay, the required number of standard vials are removed and allowed to equilibrate to room temperature.

Injection of each standard is made in triplicate prior to the injection of any test solution. At suitable intervals during the processing of test solutions, repeat injections of standards are made. In the absence of reliable CBDA and Δ⁹ THCA standards, these compounds are analysed using, respectively, the CBD and Δ⁹ THC standard response factors.

d) Test Solutions

Diluted test solutions are made up in methanol and should contain analytes in the linear working range of 0.02-0.2 mg/ml.

e) Chromatography Acceptance Criteria:

The following acceptance criteria are applied to the results of each sequence as they have been found to result in adequate resolution of all analytes (including the two most closely eluting analytes CBD and CBDA)

TABLE 1
Retention time windows and Relative Retention
Time (RRT) to Δ⁹ THC for each analyte

| | Retention time | |
|---|---|---|
| Cannabinoid | (minutes) | RRT (THC) |
| CBD | 5.1-5.8 | 0.58 |
| CBN | 7.4-8.3 | 0.83 |
| Δ⁹ THC | 9.0-10.0 | 1.00 |
| CBDA | 5.5-6.2 | 0.615 |
| Δ⁹ THCV | 5.9-6.2 | 0.645 |
| CBC | 11.6-12.8 | 1.30 |
| Δ⁹ THCA | 14.6-16.0 | 1.605 |

TABLE 2
Peak Shape (Symmetry Factor according
to British Pharmacopoeia method)

| Cannabinoid | Symmetry factor |
|---|---|
| CBD | <1.30 |
| CBN | <1.25 |
| Δ⁹ THC | <1.35 |

f) Data Processing

Cannabinoids can be subdivided into neutral and acidic-the qualitative identification can be performed using the DAD dual wavelength mode. Acidic cannabinoids absorb strongly in the region of 220 nm-310 nm. Neutral cannabinoids only absorb strongly in the region of 220 nm.

Routinely, only the data recorded at 220 nm is used for quantitative analysis.

The DAD can also be set up to take UV spectral scans of each peak, which can then be stored in a spectral library and used for identification purposes.

Data processing for quantitation utilises batch processing software on the Hewlett Packard Chemstation.

g) calculation:

Chromatographic purity of cannabinoid samples is calculated as a % of total cannabinoid content by area normalization.

Capillary Gas Chromatography (GC) Analysis

a) Chromatography Equipment and Conditions

Equipment Agilent (HP) 5890 or 6890 GLC system with HP7673 Autosampler and FID detector

GLC column SE54(ECS) 30 m×0.32 mm i.d. (Alltech) phase thickness 0.25 μm

Flow rate Constant pressure (10.3 psi). Normal initial flow rate 34 cm sec⁻¹ (2.0 ml min⁻¹)

Column oven 70° C. initially then ramp 5° C. min⁻¹ , to 250° C. Hold at 250° C. for 15 minutes.

Injector temp 250° C.

Detector temp 325° C.

Injection Vol 1 μl, split ratio 2.5:1

Run time 45 minutes

Fuel gases Hydrogen 40 ml min⁻¹

Air 450 ml min⁻¹

Helium 45 ml min⁻¹

b) Standard Preparation

Stock standard solutions of CBD, CBN and Δ⁹ THC in methanol at approximately 1 mg/ml are stored at −20° C.

Diluted working standards (0.1 mg/ml for Δ9 THC and CBD and 0.01 mg/ml for CBN) are prepared in methanol from the stock standards and stored at −20° C. (maximum period of twelve months after initial preparation). Allow an aliquot pipetted into an autosampler vial to equilibriate to room temperature prior to use in a GC assay.

c) Sample Preparation

Samples of final products, i.e. “pure” cannabidiol, are diluted in methanol prior to HPLC analysis. Optimal dilutions may be determined empirically.

Cannabis plant material samples are prepared by taking 100 mg chopped dried material and treating this with 5 or 10 ml of Methanol/Chloroform (9:1 v/v). Extract the sample in an ultrasonic bath for 15 minutes and allow to stand in the dark for 18 hours.

d) Chromatography Procedure

Standard solutions are used to provide quantitative and retention time data. These can be typically injected in triplicate prior to the injection of any sample solutions and then singularly at suitable intervals during the run, with a maximum of 10 test samples in between standards.

TABLE 3
Retention times

| | |
|---|---|
| THCV | 33.7-34.5 minutes |
| CBD | 35.6-36.3 minutes |
| Δ⁹ THC | 37.2-38.1 minutes |
| CBN | 38.5-39.1 minutes |

TLC Analysis

The qualitative composition of final products and starting materials may also be monitored by TLC.

TLC uses both retention time and characteristic spot colour to effectively identify the cannabinoid/cannabinoid acid components in a complex mixture. Methanolic solutions of the final products and starting material, plus standards, are prepared for TLC. An aliquot is spotted onto a TLC plate, alongside suitable reference samples (e.g. for at least Δ⁹ THC and CBD). Following exposure to Fast Blue B reagent, THC and THCA present as pink spots, while CBD and CBDA are orange in colour. Neutrals can be distinguished from the acids by comparison of the Rf value to that obtained for the standards. Identity is confirmed by comparison of Rf and colour of the sample spot, to that obtained for the appropriate standard.

A typical TLC protocol is as follows:

a) Materials and methods

Equipment:

Application device capable of delivering an accurately controlled volume of solution i.e 1 μl capillary pipette or micro litre syringe.

TLC development tank with lid

Hot air blower

Silica gel G TLC plates (SIL N-HR/UV254), 200 μm layer with fluorescent indicator on polyester support.

Dipping tank for visualisation reagent.

Mobile phase 80% petroleum ether 60:80/20% Diethyl ether.

Visualisation reagent 0.1% w/v aqueous Past Blue B salt BN (Sigma Corp) (100 mg in 100 ml de-ionised water). An optional method is to scan at UV 254 and 365 nm.

b) Sample Preparation

i) Herbal Raw Material

Approximately 200 mg of finely ground, dried cannabis is weighed into a 10 ml volumetric flask. Make up to volume using methanol:chloroform (9:1) extraction solvent.

Extract by ultrasound for 15 minutes. Decant supernatant and use directly for chromatography.

ii) Final Products

The final products (crystalline CBD) are dissolved in methanol to a suitable concentration (which may be determined empirically) then used directly for chromatography. All sample preparations should produce a final concentration of about 0.5 mg/ml.

iii) Botanical Drug Substance

Accurately weigh approximately 50 mg of botanical drug substance into a 25 ml volumetric flask. Dissolve to make volume with HPLC grade methanol.

c) Standards

0.1 mg/ml Δ⁹-THC in methanol (Sigma).

0.1 mg/ml CBD in methanol (Sigma).

The standard solutions are stored frozen at −20° C. between uses and are used for up to 12 months after initial preparation:

d) Test Solutions and Method

Apply to points separated by a minimum of 10 mm.

i) either 5 μl of herb extract or 1 μl of pure cannabinoid/enriched extract solution or 1 μl of diluted column eluate as appropriate,

ii) 5 μl of 0.1 mg/ml Δ⁹-THC in methanol standard solution,

iii) 5 μl of 0.1 mg/ml CBD in methanol standard solution.

Dry the prepared plate with a hot air blower.

Place the base of the TLC plate in a development tank containing the mobile phase and saturated with vapour.

Elute the TLC plate through a distance of 8 cm, then remove the plate. Allow solvent to evaporate from the plate and then repeat the elution for a second time (double development). Remove plate and allow it to dry in air.

The entire plate is briefly immersed in the Fast Blue B reagent until the characteristic red/orange colour of cannabinoids begins to develop. The plate is removed and allowed to dry under ambient conditions in the dark.

Cannabinoids will give an orange-purple colour:

| | | |
|---|---|---|
| Cannabidiol | CBD | orange (fastest running) |
| Δ⁹ Tetrahydrocannabinol | THC | pink |
| Cannabinol | CBN | purple |
| Cannabichromene | CBC | pink purple |
| Cannabigerol | CBG | orange |
| Δ⁹ tetrahydrocannabivarin | THCV | purple |

The corresponding acids form streaks of the same colour as the neutral component spots. The acids run at lower R_{f}.

1. A method of obtaining substantially pure cannabidiol (CBD) from plant material, which has a purity of greater than 95% as determined by area normalisation of an HPLC profile, which method comprises obtaining a cannabidiol-containing extract of the plant material, dissolving the extract in a solvent to form a solution, removing insoluble material from this solution and evaporating the solvent from the solution to obtain substantially pure cannabidiol, wherein the solvent is a C5-C12 straight chain or branched alkane or a carbonate ester of a C1-C12 alcohol.

2. A method according to claim 1 wherein the substantially pure preparation of cannabidiol (CBD) has a chromatographic purity of 98% or greater, preferably 99% or greater, and most preferably 99.5% or greater by area normalisation of an HPLC profile.

3. A method according to claim 2 wherein the substantially pure preparation of cannabidiol has a melting point in the range of from 64 to 66° C.

4. A method according to claim 2 wherein the substantially pure preparation of cannabidiol comprises less than 1%, preferably less than 0.8%, more preferably less than 0.6%, more preferably less than 0.4%, more preferably less than 0.2% and most preferably less than 0.1% Δ⁹ THC.

5. A method according to claim 1 wherein the insoluble material is removed by filtration.

6. A method according to claim 1 wherein the solvent is pentane, hexane or propyl carbonate.

7. A method according to claim 6 wherein the solvent is pentane.

8. A method according to claim 1 wherein the cannabidiol-containing extract of the plant material is a botanical drug substance (BDS) derived from the plant material.

9. A method according to claim 8 wherein the botanical drug substance is prepared by a process comprising solvent extraction of the plant material.

10. A method according to claim 9 wherein the botanical drug substance is prepared by extraction with carbon dioxide, ethanol, methanol or hexane.

11. A method according to claim 10 wherein the botanical drug substance is prepared by a process comprising extraction with carbon dioxide (CO₂), followed by a secondary extraction step to remove a proportion of the non-target materials.

12. A method according to claim 11 wherein the secondary extraction step is ethanolic precipitation.

13. A method according to claim 11 which further includes a charcoal clean-up step.

14. A method according to claim 13 wherein the botanical drug substance is prepared by a process comprising:
i) decarboxylation of the plant material,
ii) extraction with liquid CO₂, to produce a crude botanical drug substance,
iii) precipitation with C1-C5 alcohol to reduce the proportion of non-target materials,
iv) removal of the precipitate,
v) treatment of the resulting solution with activated charcoal, and
vi) removal of C1-C5 alcohol and water, thereby producing a final botanical drug substance.

15. A method of obtaining substantially pure cannabidiol (CBD) from plant material comprising:
i) decarboxylation of the plant material,
ii) extraction with liquid CO₂, to produce a crude botanical drug substance,
iii) precipitation with ethanol to reduce the proportion of non-target materials,
iv) filtration to remove the precipitate,
v) treatment of the resulting solution with activated charcoal,
vi) removal of ethanol and water from the solution to produce a CBD-enriched extract,
vii) re-dissolving the CBD-enriched extract in a C5-C12 straight chain or branched alkane or a carbonate ester of a C1-C12 alcohol, and
vi) removal of solvent from the solution of step vii) to obtain substantially pure CBD.

16. A method according to claim 15 wherein the solvent of step v) is pentane.

17. A method according to claim 1 wherein the substantially pure cannabidiol is obtained in crystalline form.

18. A substantially pure preparation of cannabidiol (CBD) prepared from plant material using the method of claim 1, having a chromatographic purity of 98% or greater, preferably 99% or greater, and most preferably 99.5% or greater by area normalisation of an HPLC profile.

19. A substantially pure preparation of cannabidiol according to claim 18 which is a white crystalline solid at room temperature.

20. A substantially pure preparation of cannabidiol according to claim 19 which has a melting point in the range of from 64 to 66° C.

21. A substantially pure preparation of cannabidiol according to claim 18 which comprises less than 1%, preferably less than 0.8%, more preferably less than 0.6%, more preferably less than 0.4%, more preferably less than 0.2% and most preferably less than 0.1% Δ⁹ tetrahydrocannabinol (THC).

22. A substantially pure preparation of cannabidiol according to claim 18 which comprises less than 1%, preferably less than 0.8%, more preferably less than 0.6%, more preferably less than 0.4%, more preferably less than 0.2% and most preferably less than 0.1% cannabinol (CBN).

23. A substantially pure preparation of cannabidiol according to claim 18 which is obtained from cannabis plant material using a method comprising:
i) decarboxylation of the plant material,
ii) extraction with liquid CO₂, to produce a crude botanical drug substance,
iii) precipitation with ethanol to reduce the proportion of non-target materials,
iv) filtration to remove the precipitate,
v) treatment of the resulting solution with activated charcoal,
vi) removal of ethanol and water from the solution to produce a CBD-enriched extract,
v) re-dissolving the CBD-enriched extract in pentane, and
vi) removal of pentane from the solution of step v) to obtain substantially pure CBD.

24. (canceled)

25. A substantially pure preparation of cannabidiol having an HPLC profile with a CBD retention time of 5.1-5.8 minutes.
